(19) United States
(12) Patent Application Publication
(10) Pub. No.: US 2015/0075529 A1
(43) Pub. Date: Mar. 19, 2015

(54) SYSTEM AND METHOD FOR PRESSURE SUPPORT THERAPY WITH SHAPED AIRFLOW

(71) Applicant: KONINKLIJKE PHILIPS N.V., EINDHOVEN (NL)

(72) Inventor: Robert Romano, Pittsburgh, PA (US)

(21) Appl. No.: 14/396,187

(22) PCT Filed: May 21, 2013
(86) PCT No.: PCT/IB2013/054165

§ 371 Date: Oct. 22, 2014

Related U.S. Application Data
(60) Provisional application No. 61/649,996, filed on May 22, 2012.

(51) Int. Cl. A61M 16/00; A61M 16/06; A61M 16/20; A61M 16/04

(52) U.S. Cl. CPC A61M 16/0069; A61M 16/0003; A61M 16/04; A61M 16/06; A61M 16/0666; A61M 16/20; A61M 2016/003; A61M 2205/3334; A61M 2205/505
USPC 128/204.21; 128/204.18

(57) ABSTRACT

The present disclosure pertains to an airflow shaping system (10) configured to deliver a shaped, pressurized flow of breathable gas to the airway of a subject (12). In some embodiments, the system comprises a pressure generator (14), a subject interface (16), a flow shaper (18), one or more sensors (20), one or more processors (22), electronic storage (24), a user interface (26), and/or other components. Respiratory therapy delivered to a patient's lungs may be enhanced with a shaped flow. A turbulent flow of gas delivered to a patient's lungs is inefficient in the amount of energy required to deliver the gas and because it requires more gas energy to reach the lower airways due to resistance from the airway walls. The system may be used for respiratory therapy applications including pressure support therapy, inexsufflation therapy, airway medication delivery, and/or other applications.

The present disclosure pertains to an airflow shaping system configured to deliver a shaped, pressurized flow of breathable gas to the airway of a subject.

Systems for providing respiratory therapy (e.g., positive airway pressure, inexsufflation, etc.) to subjects are known. These systems generate a pressurized flow of breathable gas that is provided to the airway of a subject to support the subject's airway. In one type of positive airway pressure (PAP) therapy, known as continuous positive air pressure (CPAP), the pressure of gas delivered to the patient is constant throughout the patient's breathing cycle. It is also known to provide a positive pressure therapy in which the pressure of gas delivered to the patient varies with the patient's breathing cycle, or varies with the patient's effort, to increase the comfort to the patient. During these conventional pressure support therapy techniques the airflow is typically chaotic and/or turbulent.

Accordingly, one or more aspects of the present disclosure relate to an airflow shaping system configured to deliver a shaped, pressurized flow of breathable gas to the airway of a subject. The system comprises a pressure generator, a subject interface, and a flow shaper. The pressure generator is configured to generate a flow of breathable gas for delivery to an airway of the subject. The subject interface is configured to place the pressure generator in fluid communication with the airway of the subject. The flow shaper is configured to impart a delivery shape to the flow of breathable gas such that the flow of gas reaches the airway of the subject through the subject interface with the delivery shape imparted by the flow shaper.

Yet another aspect of the present disclosure relates to a method of delivering a shaped, pressurized flow of breathable gas to an airway of a subject with an airflow shaping system. The airflow shaping system comprises a pressure generator, a subject interface, and a flow shaper. The method comprises generating the pressurized flow of breathable gas with the pressure generator; communicating the pressurized flow of breathable gas to the airway of the subject with the subject interface; and imparting a delivery shape to the pressurized flow of breathable gas with the flow shaper such that the flow of gas reaches the airway of the subject through the subject interface with the delivery shape imparted by the flow shaper.

Still another aspect of the present disclosure relates to an airflow shaping system configured to deliver a shaped, pressurized flow of breathable gas to the airway of a subject. The system comprises means for generating the pressurized flow of breathable gas; means for communicating the pressurized flow of breathable gas to the airway of the subject; and means for imparting a delivery shape to the pressurized flow of breathable gas such that the flow of gas reaches the airway of the subject through the means for communicating with the delivery shape imparted by the means for imparting.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

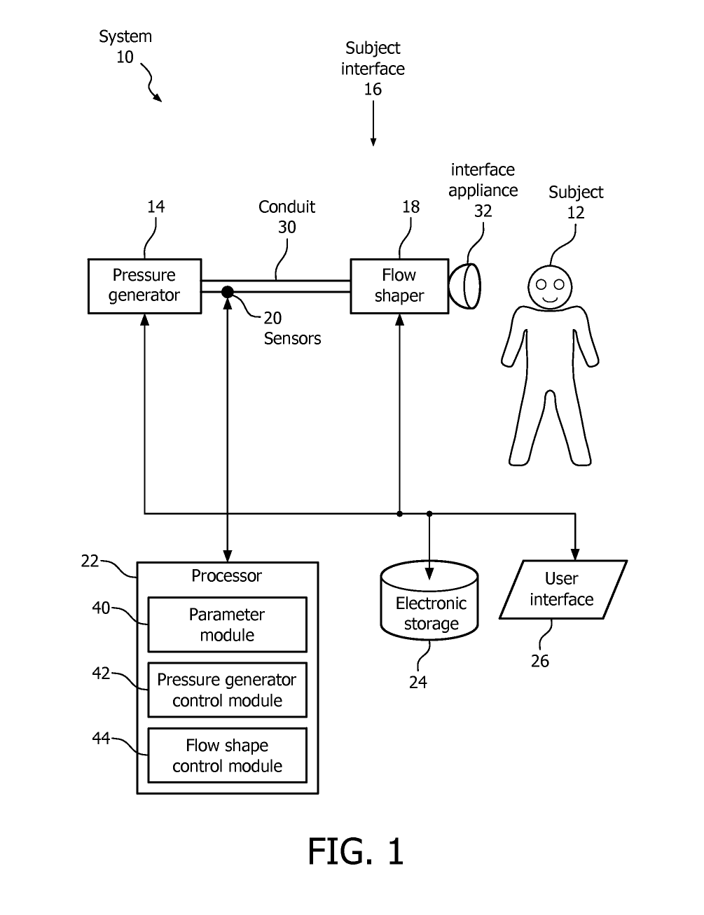

FIG. 1 schematically illustrates an exemplary embodiment of a system configured to deliver a shaped, pressurized flow of breathable gas to the airway of a subject;

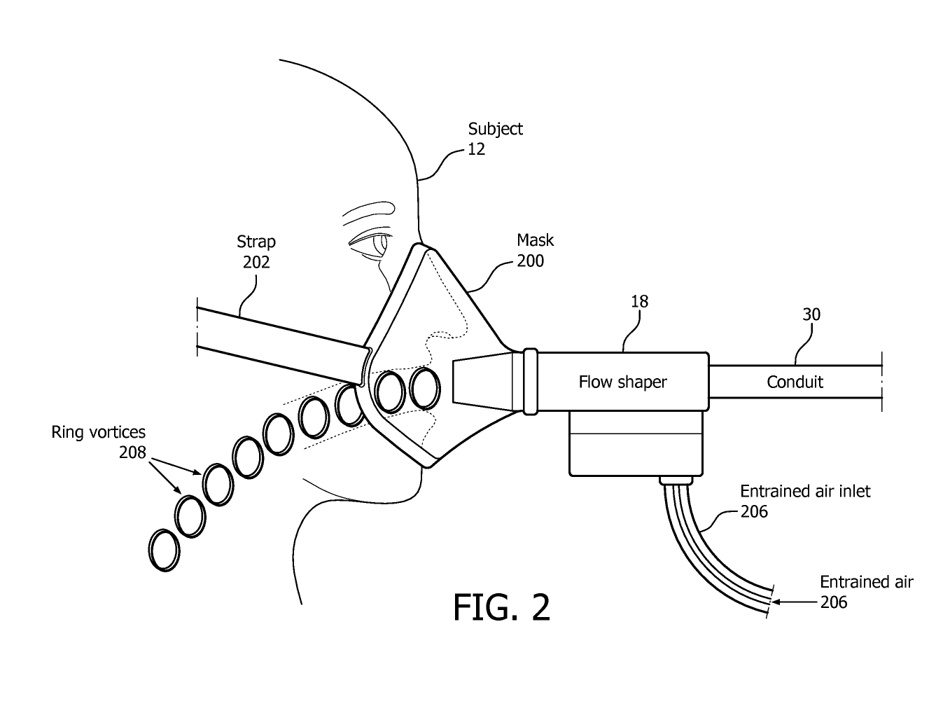

FIG. 2 shows a flow shaper coupled to a mask;

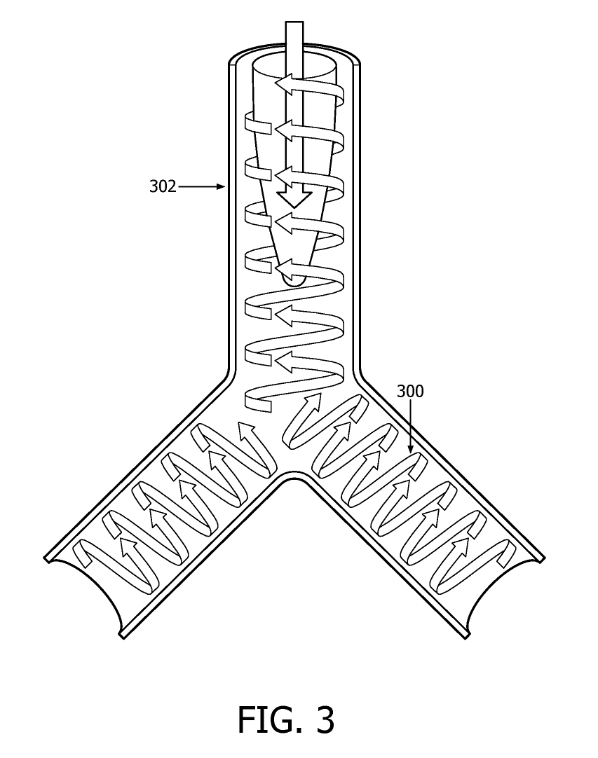

FIG. 3 depicts cyclonic airflow in the airway of a subject; and

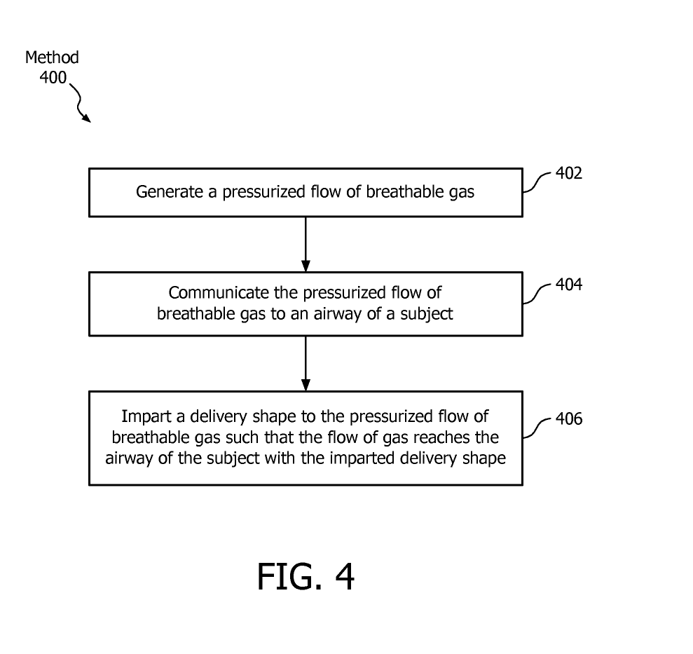

FIG. 4 illustrates a method of delivering a shaped, pressurized flow of breathable gas to an airway of a subject with an airflow shaping system.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIG. 1 schematically illustrates an exemplary embodiment of a system 10 configured to deliver a shaped, pressurized flow of breathable gas to the airway of a subject 12. In some embodiments, system 10 comprises a pressure generator 14, a subject interface 16, a flow shaper 18, one or more sensors 20, one or more processors 22, electronic storage 24, a user interface 26, and/or other components.

Respiratory therapy delivered to a patient's lungs may be enhanced with a shaped flow of gas. A turbulent flow of gas delivered to a patient's lungs is inefficient in the amount of energy required to deliver the gas and because it requires more gas energy to reach the lower airways due to resistance from the airway walls. A laminar flow profile is difficult to administer and maintain unless the gas is delivered directly into the airway by invasive means such as a cannula via a transtracheal puncture or endotracheal tube. Gas may be delivered through a convenient non-invasive appliance (e.g., a face mask) with a shaped flow in the form of, for example, toroidal ring vortices, a cyclonic flow, and/or other forms. The result may have several potential benefits including improved gas exchange (recruitment of atelectatic areas, improved oxygenation, minimization of dead space), improved delivery of medications by transporting the aerosolized medication deeper into the lower airways and alveoli regions of the lungs, improvement of the mucociliary transport system, and reduced risk of barotraumas.

System 10 may be used for respiratory therapy applications including pressure support therapy, inexsufflation therapy, airway medication delivery, and/or other applications.

Pressure generator 14 is configured to provide a pressurized flow of breathable gas for delivery to the airway of subject 12 according to a respiratory therapy regime. The respiratory therapy regime may include one or more of non-invasive ventilation (NIV), CPAP, bi-level positive airway pressure (BPAP), proportional positive airway pressure support (PPAP), inexsufflation therapy, and/or other respiration therapy regimes. Pressure generator 14 receives a flow of gas from a gas source, such as the ambient atmosphere, and elevates the pressure of that gas for delivery to subject 12. In some embodiments, pressure generator 14 is configured to generate negative pressure to draw gas from subject 12. Pressure generator 14 may be configured such that one or more gas parameters of the pressurized flow of breathable gas are controlled in accordance with the therapy regime. The one or more gas parameters may include, for example, one or more of pressure, volume, flow rate, temperature, gas composition, velocity, acceleration, and/or other parameters.

In some embodiments, pressure generator 14 may include any device, such as, for example, a pump, blower, piston, or bellows, that is capable of elevating the pressure of the received gas for delivery to a patient. In some embodiments, pressure generator 14 may include one or more devices, such as for example, a valve and/or a series of valves, capable of controlling the pressure, flow rate, flow direction, and/or other parameters of the flow of gas. The present disclosure contemplates controlling the operating speed of the blower, for example, either alone or in combination with one or more valves and/or other devices contained in and/or external to pressure generator 14, to control the pressure and/or flow of gas provided to subject 12. For example, pressure generator 14 may selectively control the pressure of the flow of gas such that an inspiratory positive airway pressure (IPAP) delivered to the patient is higher than an expiratory positive airway pressure (EPAP) during bi-level pressure support. The present disclosure contemplates that gas other than ambient atmospheric air may be introduced into system 10 for delivery to the patient.

Subject interface 16 is configured to interface with the airway of subject 12. Subject interface 16 is configured to provide fluid communication between pressure generator 14 and the airway of subject 12. As such, subject interface 16 comprises a conduit 30, an interface appliance 32, and/or other components. Conduit 30 is configured to form a flow path through which the pressurized flow of breathable gas is communicated between pressure generator 14 and interface appliance 32. Conduit 30 may be a flexible length of hose, or other conduit, that places interface appliance 32 in fluid communication with pressure generator 14. Conduit 30 conveys gas (e.g., air) to and/or from interface appliance 32, and interface appliance 32 places conduit 30 in communication with the airway of subject 12. In some embodiments, interface appliance 32 is non-invasive. As such, interface appliance 32 non-invasively engages subject 12. Non-invasive engagement includes removably engaging an area (or areas) surrounding one or more external orifices of the airway of subject 12 (e.g., nostrils and/or mouth) to communicate gas between the airway of subject 12 and subject interface 16. Some examples of non-invasive interface appliance 32 may include, for example, a blow tube, a nasal cannula, a nasal mask, a nasal/oral mask, a full face mask, a total face mask, or other interface appliances that communicate a flow of gas with an airway of a subject.

Flow shaper 18 is configured to impart a delivery shape to the flow of breathable gas such that the flow of gas reaches the airway of the subject through subject interface 16 with the delivery shape imparted by flow shaper 18. The delivery shape imparted by flow shaper 18 may refer to geometrical information about a form factor of the flow of gas, timing information (e.g., frequency), scale, rotational information, appearance, physical qualities (e.g., density), and/or other information. In some embodiments, flow shaper 18 may be configured to continuously shape the flow of gas for delivery to subject 12 during a respiratory therapy session. In some embodiments, flow shaper 18 may be configured to shape the flow of gas into a series of one or more air boluses. By way of a non-limiting example, flow shaper 18 may impart a delivery shape to the flow of breathable gas such that the flow of gas reaches the airway of subject 12 configured as a series of ring vortices (toroidal shape). The ring vortices may be formed when the breathable gas passes (e.g., configured as a series of air boluses) through an aperture in flow shaper 18 and pushes up against non-flowing, substantially calm gas in conduit 30, interface appliance 32, or another component of system 10. The flow of gas in the ring vortex may rotate about the circular axis of the ring. By way of another non-limiting example, the shape imparted by flow shaper 18 may comprise cyclonic air flow in which the air flows in a helical pattern.

In some embodiments, flow shaper 18 may include components, such as, for example, mechanical components, electromechanical components, a spinning disk, apertures, a diaphragm, vanes, and/or other components capable of shaping the flow of gas for delivery to subject 12. In some embodiments, the flow of gas from pressure generator 14 may be disrupted by the spinning disk, apertures, vanes, and/or other components to impart a shape to the flow of gas. In some embodiments, flow shaper 18 may be configured with an oscillating membrane that forces gas through an aperture. In some embodiments, flow shaper 18 may include one or more devices, such as for example, a valve and/or a series of valves, capable of controlling the pressure, flow rate, and/or other parameters of the shaped flow of gas. The present disclosure contemplates controlling the shaping components of flow shaper 18 either alone or in combination with the one or more valves and/or other devices contained in and/or external to flow shaper 18, to control the shape and/or flow of the gas delivered to subject 12 (e.g., air boluses).

FIG. 1 shows flow shaper 18 located in subject interface 16 between pressure generator 14 and interface appliance 32, coupled to conduit 30 on one side and interface appliance 32 on the other. The location of flow shaper 18 in interface appliance 16 is not intended to be limiting. Pressure generator 14 may be directly coupled to flow shaper 18. Pressure generator 14 may be included in flow shaper 18 such that pressure generation and flow shaping are performed by the same device. Flow shaper 18 may be directly coupled interface appliance 32. Pressure generator 14, flow shaper 18, interface appliance 32, and/or other components may be coupled such that conduit 30 need not be included in system 10. In short, any configuration of the components of system 10 that allow system 10 to function as described herein is contemplated by the present disclosure.

By way of a non-limiting example, FIG. 2 shows flow shaper 18 coupled to a mask 200. The mask is attached to the face of subject 12 with a strap 202. Flow shaper 18 may receive gas through conduit 30. Flow shaper 18 may entrain additional air 204 via an entrained air inlet 206. Entraining additional air may, for example, reduce the amount of source gas (e.g., oxygen) needed to deliver therapeutic levels for effective pulmonary gas exchange. In FIG. 2, flow shaper 18 shaped the flow of gas into a series of ring vortices 208. Flow shaper 18 shaped the ring vortices such that the flow of gas reaches the airway of subject 12 through mask 200 with the ring vortex delivery shape imparted by flow shaper 18.

In some embodiments, flow shaper 18 may be configured to generate and/or shape the pressurized flow of breathable gas for delivery to the airway of subject 12. As such, flow shaper 18 may receive a flow of gas from a gas source (e.g., the ambient atmosphere, an oxygen cylinder) and elevate the pressure of that gas for delivery to subject 12. In the example configuration shown in FIG. 2, flow shaper 18 may be configured to draw gas in through conduit 30 and/or entrained air inlet 206. Flow shaper 18 may include any device, such as, for example, a pump, blower, piston, or bellows, that is capable of elevating the pressure of the received gas for delivery to a patient.

By way of a second non-limiting example, FIG. 3 depicts cyclonic airflow 300 in the airway 302 of a subject. Cyclonic airflow is a high speed rotating airflow established within a cylindrical or conical container. Flow shaper 18 (not shown in FIG. 3) may be configured to generate cyclonic airflow by directing the flow of air around the circumference of a cylinder and/or cone within flow shaper 18 such that the air flows in a helical pattern.

Returning to FIG. 1, sensors 20 are configured to generate output signals conveying information related to one or more gas parameters of the gas within subject interface 16. The one or more gas parameters may comprise flow rate, pressure, volume, temperature, humidity, velocity, and/or other gas parameters. Sensors 20 may comprise one or more sensors that measure such parameters directly (e.g., through fluid communication with the flow of gas in subject interface 16). Sensors 20 may comprise one or more sensors that generate output signals related to one or more parameters of the flow of gas indirectly. By way of a non-limiting example, one or more of sensors 20 may generate an output based on an operating parameter of the pressure generator 14 (e.g., a motor current, voltage, rotational velocity, and/or other operating parameters), and/or other sensors. Although sensors 20 are illustrated at a single location within (or in communication with) conduit 30 between flow shaper 18 and pressure generator 14, this is not intended to be limiting. Sensors 20 may include sensors disposed in a plurality of locations, such as for example, within pressure generator 14, within flow shaper 18, within (or in communication with) interface appliance 32, and/or other locations.

Processor 22 is configured to provide information processing capabilities in system 10. As such, processor 22 may include one or more of a digital processor, an analog processor, a digital circuit designed to process information, an analog circuit designed to process information, a state machine, and/or other mechanisms for electronically processing information. Although processor 22 is shown in FIG. 1 as a single entity, this is for illustrative purposes only. In some implementations, processor 22 includes a plurality of processing units. These processing units may be physically located within the same device, or processor 22 may represent processing functionality of a plurality of devices operating in coordination.

As shown in FIG. 1, processor 22 may be configured to execute one or more computer program modules. The one or more computer program modules comprise one or more of a parameter module 40, a pressure generator control module 42, a flow shape control module 44, and/or other modules. Processor 22 may be configured to execute modules 40, 42, and/or 44 by software; hardware; firmware; some combination of software, hardware, and/or firmware; and/or other mechanisms for configuring processing capabilities on processor 22.

It should be appreciated that although modules 40, 42, and 44 are illustrated in FIG. 1 as being co-located within a single processing unit, in implementations in which processor 22 includes multiple processing units, one or more of modules 40, 42, and/or 44 may be located remotely from the other modules. The description of the functionality provided by the different modules 40, 42, and/or 44 described below is for illustrative purposes, and is not intended to be limiting, as any of modules 40, 42, and/or 44 may provide more or less functionality than is described. For example, one or more of modules 40, 42, and/or 44 may be eliminated, and some or all of its functionality may be provided by other ones of modules 40, 42, and/or 44. As another example, processor 22 may be configured to execute one or more additional modules that may perform some or all of the functionality attributed below to one of modules 40, 42, and/or 44.

Parameter module 40 is configured to determine one or more parameters within system 10. The one or more parameters within system 10 may comprise gas parameters related to the pressurized flow of breathable gas, breathing parameters related to the respiration of subject 12, shape parameters related to the shape of the flow of gas delivered to subject 12, and/or other parameters. Parameter module 40 is configured to determine the one or more parameters based on the output signals of sensors 20, and/or other information. The information determined by parameter module 40 may be used for controlling pressure generator 14, controlling flow shaper 18, stored in electronic storage 24, displayed by user interface 26, and/or used for other uses. The one or more parameters determined by parameter module 40 may comprise, for example, one or more of a flow rate, flow shape, pressure, a volume, humidity, temperature, acceleration, velocity, respiration rate, tidal volume, and/or other parameters.

Pressure generator control module 42 is configured to control pressure generator 14 to generate the flow of gas in accordance with the respiratory therapy regime. The respiratory therapy regime may include one or more of non-invasive ventilation (NIV), CPAP, BPAP, proportional positive airway pressure support (PPAP), inexsufflation therapy, and/or other respiration therapy regimes. Pressure generator control module 42 is configured to control pressure generator 14 based on information related to the output signals from sensors 20, information determined by parameter module 40, information entered by a user to user interface 26, and/or other information.

Flow shape control module 44 is configured to control flow shaper 18. Flow shape control module 44 is configured to control flow shaper 18 to control one or more of the form factor, pressure, volume, frequency, velocity, and/or other parameters of the shape provided by flow shaper 18. Flow shape control module 44 is configured to control flow shaper 18 based on information related to the output signals from sensors 20, information determined by parameter module 40, information entered by a user to user interface 26, and/or other information.

Flow shape control module 44 may be configured to control flow shaper 18 to superimpose the imparted shape on the flow of gas generated by pressure generator 14 (e.g., continuous positive airway pressure support, bi-level pressure support, inexsufflation therapy, etc.). Superimposing the imparted shape on the flow of gas generated by pressure generator 14 may comprise imparting the shape to the flow of gas such that the characteristics (e.g., pressure during inhalation versus pressure during exhalation) of the flow of gas that are related to the therapy regime are still evident. For example, pressure generator 14 may selectively control the pressure of the flow of gas such that an inspiratory positive airway pressure (IPAP) delivered to the patient is higher than an expiratory positive airway pressure (EPAP) during bi-level pressure support. Flow shape control module 44 may control flow shaper 18 to superimpose cyclonic air flow during respiration such that the cyclonic airflow is delivered at the inspiratory pressure and the expiratory pressure generated by pressure generator 14.

In some embodiments, flow shape control module 44 may be configured to control flow shaper 18 to continuously shape the flow of gas for delivery to subject 12 during a therapy session (e.g., bi-level cyclonic airflow described above). In some embodiments, flow shape control module 44 may control flow shaper 18 to shape the flow of gas intermittently (e.g., only during inspiration, during a portion of inspiration, during a portion of inspiration and a portion of expiration, etc.).

In some embodiments, flow shape control module 44 may be configured to control flow shaper 18 to shape the flow of gas into a series of one or more air boluses. Flow shape control module 44 may control flow shaper 18 to deliver the boluses of gas continuously and/or intermittently during a therapy session. Flow shape control module 44 may control flow shaper 18 to control the air bolus form factor, volume, pressure, frequency, and/or other parameters. In some embodiments, flow shape control module 44 may control flow shaper 18 such that the air boluses have a pressure of up to about 5 cm H₂O. In some embodiments, flow shape control module 44 may control flow shaper 18 such that the air boluses have a pressure of up to about 4 cm H₂O. In some embodiments, flow shape control module 44 may control flow shaper 18 such that the air boluses have a pressure of up to about 3 cm H₂O. In some embodiments, flow shaper 18 may be configured to deliver air boluses to the airway of subject 12 at a frequency up to 400 boluses per minute. In some embodiments, flow shaper 18 may be configured to deliver the air boluses to the airway of subject 12 at a frequency up to 300 boluses per minute. In some embodiments, flow shaper 18 may be configured to deliver the air boluses to the airway of subject 12 at a frequency up to 200 boluses per minute.

In some embodiments, flow shape control module 44 may control flow shaper 18 such that delivery of the air boluses generates a percussive pressure waveform. The percussive pressure waveform may be generated by abruptly alternating the pressure delivered to subject 12 between the bolus pressure and one or more other pressure levels, controlling the frequency of bolus delivery, and/or or by another method.

In some embodiments, electronic storage 24 comprises electronic storage media that electronically stores information. The electronic storage media of electronic storage 24 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 24 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 24 may store software algorithms, information determined by processor 22, information received via user interface 26, and/or other information that enables system 10 to function properly. Electronic storage 24 may be (in whole or in part) a separate component within system 10, or electronic storage 24 may be provided (in whole or in part) integrally with one or more other components of system 10 (e.g., pressure generator 14, processor 22, etc.). In some embodiments, information determined by processor 22 and stored by electronic storage 24 may comprise information related to previous respiration by subject 12, information related to previous respiration therapy on subject 12 by system 10, information related to flow shaper 18 and/or other information.

User interface 26 is configured to provide an interface between system 10 and subject 12 through which subject 12 provides information to and receives information from system 10. This enables data, results, and/or instructions and any other communicable items, collectively referred to as “information,” to be communicated between subject 12 and one or more of subject interface 16, processor 22, and/or other components of system 10. Examples of interface devices suitable for inclusion in user interface 26 include a keypad, buttons, switches, a keyboard, knobs, levers, a display screen, a touch screen, speakers, a microphone, a printer, and/or other interface devices. In some embodiments, user interface 26 includes a plurality of separate interfaces. It is to be understood that other communication techniques, either hard-wired or wireless, are also contemplated by the present disclosure as user interface 26. Other exemplary input devices and techniques adapted for use with system 10 as user interface 26 include, but are not limited to, an RS-232 port, RF link, an IR link, modem (telephone, cable or other). In short, any technique for communicating information with system 10 is contemplated by the present disclosure as user interface 26. By way of a non-limiting example, a user may set the shape of the flow of gas delivered to subject 12 through user interface 26.

In some embodiments, other information entered by a user through user interface 26 to system 10 may include, for example, designation of a therapy regime (e.g., inexsufflation, CPAP, etc.), flow shape parameters (e.g., frequency, pressure, etc.), and/or other information.

FIG. 4 illustrates a method 400 of delivering a shaped, pressurized flow of breathable gas to an airway of a subject with an airflow shaping system. The airflow shaping system comprises a pressure generator, a subject interface, and a flow shaper. The operations of method 400 presented below are intended to be illustrative. In some embodiments, method 400 may be accomplished with one or more additional operations not described, and/or without one or more of the operations discussed. Additionally, the order in which the operations of method 400 are illustrated in FIG. 4 and described below is not intended to be limiting.

In some embodiments, method 400 may be implemented in one or more processing devices (e.g., a digital processor, an analog processor, a digital circuit designed to process information, an analog circuit designed to process information, a state machine, and/or other mechanisms for electronically processing information). The one or more processing devices may include one or more devices executing some or all of the operations of method 400 in response to instructions stored electronically on an electronic storage medium. The one or more processing devices may include one or more devices configured through hardware, firmware, and/or software to be specifically designed for execution of one or more of the operations of method 400.

At an operation 402, a pressurized flow of breathable gas is generated for delivery to an airway of a subject. In some embodiments, operation 402 is performed by a pressure generator the same as or similar to pressure generator 14 (shown in FIG. 1 and described herein).

At an operation 404, the pressurized flow of breathable gas is communicated to the airway of the subject with a subject interface. In some embodiments, operation 404 is performed by a subject interface the same as or similar to subject interface 16 (shown in FIG. 1 and described herein).

At an operation 406, a delivery shape is imparted to the flow of breathable gas. The delivery shape is imparted to the flow of breathable gas such that the flow of gas reaches the airway of the subject with the imparted delivery shape. In some embodiments, operation 406 is performed by a flow shaper the same as or similar to flow shaper 18 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. An airflow shaping system configured to deliver a shaped, pressurized flow of breathable gas to the airway of a subject, the system comprising:
a pressure generator configured to generate a flow of breathable gas for delivery to an airway of the subject during inhalation and exhalation;
a subject interface configured to place the pressure generator in fluid communication with the airway of the subject; and
a flow shaper configured to impart a delivery shape to the flow of breathable gas such that the airflow has a geometric shape in physical three dimensional space and the flow of gas reaches the airway of the subject through the subject interface with the delivery shape imparted by the flow shaper.

2. The system of claim 1, further comprising one or more processors configured to execute computer program modules, the computer program modules comprising a flow shape control module configured to control the flow shaper.

3. The system of claim 1, further comprising one or more sensors configured to generate one or more output signals conveying information related to one or more parameters of the flow of gas; and
one or more processors configured to execute computer program modules, the computer program modules comprising:
a pressure generator control module configured to control the pressure generator according to a therapy regime based on the output signals, wherein the pressure generator control module is configured to control the pressure generator according to one or more of a continuous positive airway pressure support therapy regime, a bi-level pressure support therapy regime, or an inexsufflation therapy regime.

4. The system of claim 3, wherein the flow shaper superimposes the imparted shape on one or more of the continuous positive airway pressure support therapy regime, the bi-level pressure support therapy regime, or the inexsufflation therapy regime.

5. The system of claim 1, wherein the shape imparted by the flow shaper comprises a cyclonic air flow, and/or a series of air vortices, and
wherein one or more individual air vortices in the series of air vortices comprises a ring vortex of gas.

6. A method of generating a shaped, pressurized flow of breathable gas for delivery to an airway of a subject with an airflow shaping system, the airflow shaping system comprising a pressure generator, a subject interface, and a flow shaper, the method comprising:
generating the pressurized flow of breathable gas with the pressure generator during inhalation and exhalation;
communicating the pressurized flow of breathable gas to the airway of the subject with the subject interface; and
imparting a delivery shape to the pressurized flow of breathable gas with the flow shaper such that the airflow has a geometric shape in physical three dimensional space and the flow of gas reaches the airway of the subject through the subject interface with the delivery shape imparted by the flow shaper.

7. The method of claim 6, further comprising executing a flow shape control module on a processor to control the imparting of the delivery shape by the flow shaper, the airflow shaping system further comprising the processor, the processor configured to execute computer program modules including the flow shape control module.

8. The method of claim 6 wherein the airflow shaping system further comprises one or more sensors and one or more processors, the method further comprising generating one or more output signals conveying information related to one or more parameters of the flow of gas with the one or more sensors, and executing computer program modules on the one or more processors, executing computer program modules comprising:
controlling generation of the flow of breathable gas according to a therapy regime based on the output signals, wherein the therapy regime comprises one or more of a continuous positive airway pressure support therapy regime, a bi-level pressure support therapy regime, or an inexsufflation therapy regime.

9. The method of claim 8, further comprising superimposing the imparted shape on one or more of the continuous positive airway pressure support therapy regime, the bi-level pressure support therapy regime, or the inexsufflation therapy regime.

10. The method of claim 6, wherein the imparted shape comprises a cyclonic air flow, and/or a series of air vortices, and
wherein one or more individual air vortices in the series of air vortices comprises a ring vortex of gas.

11. An airflow shaping system configured to deliver a shaped, pressurized flow of breathable gas to the airway of a subject, the system comprising:
means for generating the pressurized flow of breathable gas during inhalation and exhalation;
means for communicating the pressurized flow of breathable gas to the airway of the subject; and
means for imparting a delivery shape to the pressurized flow of breathable gas such that the airflow has a geometric shape in physical three dimensional space and the flow of gas reaches the airway of the subject through the means for communicating with the delivery shape imparted by the means for imparting.

12. The system of claim 11, further comprising means for executing computer program modules, the computer program modules comprising means for controlling the means for imparting a delivery shape.

13. The system of claim 11, further comprising means for generating one or more output signals conveying information related to one or more parameters of the flow of gas; and
means for executing computer program modules, the computer program modules comprising:
means for controlling the means for generating according to a therapy regime based on the output signals; and wherein the means for controlling the means for generating is configured to control the means for generating according to one or more of a continuous positive airway pressure support therapy regime, a bi-level pressure support therapy regime, or an inexsufflation therapy regime.

14. The system of claim 13, wherein the means for imparting superimposes the imparted shape on one or more of the continuous positive airway pressure support therapy regime, the bi-level pressure support therapy regime, or the inexsufflation therapy regime.

15. The system of claim 11, wherein the shape imparted by the means for imparting comprises a cyclonic air flow, and/or a series of air vortices, and
wherein one or more individual air vortices in the series of air vortices comprises a ring vortex of gas.
